(19) United States
(12) Patent Application Publication
(10) Pub. No.: US 2025/0131071 A1
(43) Pub. Date: Apr. 24, 2025

(54) CONTINUOUS RECOGNITION OF USERS THROUGH TEXT ENTRY

(71) Applicant: International Business Machines Corporation, Armonk, NY (US)

(72) Inventors: James R. Kozloski, New Fairfield, CT (US); Michael S. Gordon, Croton on Hudson, NY (US); Girmaw Abebe Tadesse, Nairobi (KE)

(21) Appl. No.: 18/490,831

(22) Filed: Oct. 20, 2023

(51) Int. Cl. G06F 21/31; G16H 50/20

(52) U.S. Cl. CPC G06F 21/316; G16H 50/20; G06F 2221/2133

(57) ABSTRACT

According to one embodiment, a method, computer system, and computer program product for human recognition through use of text entry is provided. The embodiment may include tracking user inputs. The embodiment may also include tracking time intervals between user inputs. The embodiment may further include storing input-interval sets, each input-interval set containing at least one input from the user inputs and at least one time interval from the time intervals between user inputs, wherein the at least one input corresponds to the at least one time interval. The embodiment may also include pretraining a foundation model based on the stored input-interval sets. The embodiment may further include training the foundation model for a particular task.

### BACKGROUND

The present invention relates generally to the field of computing, and more particularly to human recognition.

Human recognition is a set of techniques used to distinguish between humans and computer algorithms, and otherwise identify patterns in human behavior. Human recognition uses techniques such as CAPTCHA and natural language processing to, for example test a user's humanity, prevent spam, identify a human's emotional state or sentiment. These techniques may include presenting a user with a test, tracking patterns of users' interactions with a user interface, or training a machine learning algorithm to recognize differences between human and AI behavior.

Human recognition techniques should reliably and efficiently be able to discern between humans and algorithms without interrupting a user's experience. However, many techniques for human recognition require active input by users through bothersome user interface (“UI”) elements. Furthermore, a variety of algorithms attempt to circumvent existing techniques for human recognition, either simulating human knowledge or behavior, faking a person's identity, or using other techniques such as machine learning, visual text recognition, speech-to-text, or randomization to fool a human recognition algorithm.

### SUMMARY

According to one embodiment, a method, computer system, and computer program product for human recognition through use of text entry is provided. The embodiment may include tracking user inputs. The embodiment may also include tracking time intervals between user inputs. The embodiment may further include storing input-interval sets, each input-interval set containing at least one input from the user inputs and at least one time interval from the time intervals between user inputs, wherein the at least one input corresponds to the at least one time interval. The embodiment may also include pretraining a foundation model based on the stored input-interval sets. The embodiment may further include training the foundation model for a particular task.

In a preferred embodiment, the user inputs are characters in an input stream.

In a preferred embodiment, the intervals are tracked on a scale of milliseconds.

In a preferred embodiment, the particular task is discerning whether or not the user is a human.

In a preferred embodiment, tracking user inputs includes tracking user inputs from two or more devices.

In a preferred embodiment, the particular task is identifying or predicting a disease, disorder, or injury.

In a preferred embodiment, the method, computer system, and computer program product may further comprise performing the particular task and using feedback collected about the performance of the particular task in order to further pretrain or further train the foundation model.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment.

FIG. 2 illustrates an operational flowchart for a process for recognizing humans through text entry.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

It is to be understood that the singular forms “a,” “an,” and “the” include plural referents unless the context clearly dictates otherwise. Thus, for example, reference to “a component surface” includes reference to one or more of such surfaces unless the context clearly dictates otherwise.

Embodiments of the present invention relate to the field of computing, and more particularly to human recognition. The following described exemplary embodiments provide a system, method, and program product to, among other things, recognize patterns in human behavior. Therefore, the present embodiment has the capacity to improve the technical field of human recognition by easily, confidently, or passively checking or verifying user text patterns for a variety of tasks.

As previously described, human recognition is a set of techniques used to distinguish between humans and computer algorithms, and otherwise identify patterns in human behavior. Human recognition uses techniques such as CAPTCHA and natural language processing to, for example test a user's humanity, prevent spam, identify a human's emotional state or sentiment. These techniques may include presenting a user with a test, tracking patterns of users' interactions with a user interface, or training a machine learning algorithm to recognize differences between human and AI behavior.

Human recognition techniques should reliably and efficiently be able to discern between humans and algorithms without interrupting a user's experience. However, many techniques for human recognition require active input by users through bothersome UI elements. Furthermore, a variety of algorithms attempt to circumvent existing techniques for human recognition, either simulating human knowledge or behavior, faking a person's identity, or using other techniques such as machine learning, visual text recognition, speech-to-text, or randomization to fool human recognition algorithms. As such, it may be advantageous to use complex techniques to analyze a user's text input for tasks relating to human recognition.

According to one embodiment, a human recognition program tracks user keystrokes and intervals between keystrokes according to opt-in procedures. The human recognition program then stores a text encoding of input-interval pairs. The human recognition program then engages in pretraining of a foundation model on the input-interval pairs, and further train the pretrained model to accomplish a particular task.

One or more embodiments described above may convey the advantage of more consistently discerning between human and non-human users, or between different human users; resisting to traditional techniques of circumventing human recognition, such as visual recognition and speech-to-text; discerning between users more quickly, continuously, or passively without the need for a UI interruption; providing insights into potential motor issues or diseases that may affect user input; or providing sentiment analysis.

Any advantages listed herein are only examples and are not intended to be limiting to the illustrative embodiments. Additional or different advantages may be realized by specific illustrative embodiments. Furthermore, a particular illustrative embodiment may have some, all, or none of the advantages listed above.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

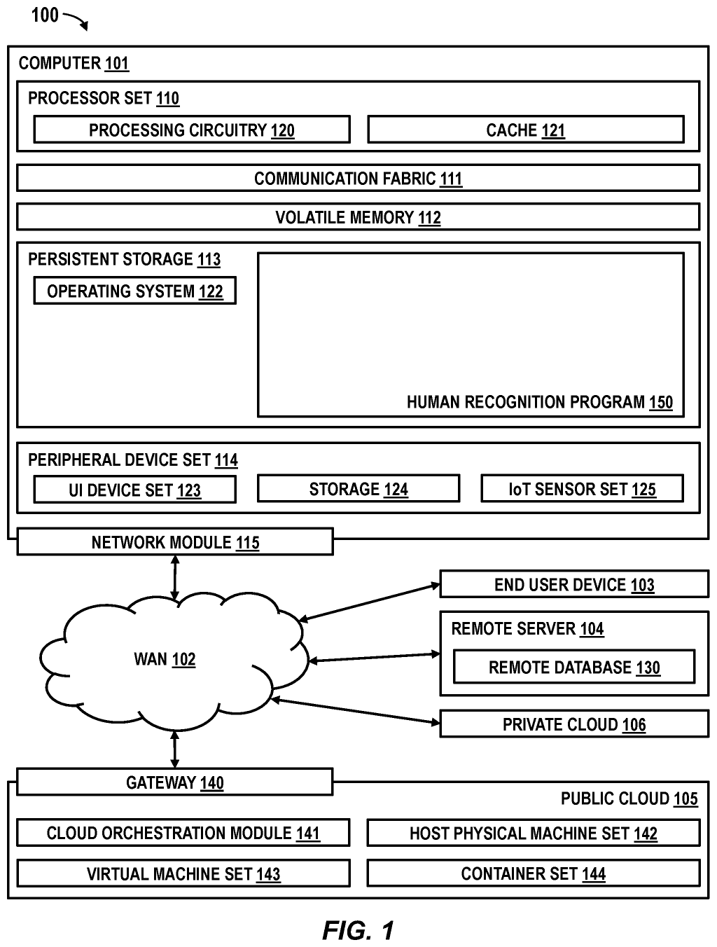

Referring now to FIG. 1, computing environment 100 contains an example of an environment for the execution of at least some of the computer code involved in performing the inventive methods, such as human recognition program 150. In addition to human recognition program 150, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and human recognition program 150, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, for illustrative brevity. Computer 101 may be located in a cloud, even though it is not shown in a cloud in FIG. 1. On the other hand, computer 101 is not required to be in a cloud except to any extent as may be affirmatively indicated.

Processor set 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in human recognition program 150 in persistent storage 113.

Communication fabric 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 112 is any type of volatile memory now known or to be developed in the future. Examples include dynamic type random access memory (RAM) or static type RAM. Typically, the volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

Persistent storage 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid-state storage devices. Operating system 122 may take several forms, such as various known proprietary operating systems or open-source Portable Operating System Interface-type operating systems that employ a kernel. The code included in human recognition program 150 typically includes at least some of the computer code involved in performing the inventive methods.

Peripheral device set 114 includes the set of peripheral devices of computer 101. Data communication connections between the peripheral devices and the other components of computer 101 may be implemented in various ways, such as Bluetooth® (Bluetooth and all Bluetooth-based trademarks and logos are trademarks or registered trademarks of the Bluetooth Special Interest Group and/or its affiliates) connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

Network module 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN 102 and/or LANs typically include computer hardware such as copper transmission cables, optical transmission fibers, wireless transmission, routers, firewalls, switches, gateway computers and edge servers.

End user device (EUD) 103 is any computer system that is used and controlled by an end user and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

Remote server 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

Public cloud 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community, or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The human recognition program 150 may track user keystrokes and intervals between keystrokes according to opt-in procedures. The human recognition program 150 may then store a text encoding of input-interval pairs. The human recognition program 150 may then pretrain a foundation model on the input-interval pairs, and further train the pretrained model for a particular task, such as discerning between a human and a machine, verifying a user's identity, emotion and sentiment analysis, or detection of motor disorders or illnesses.

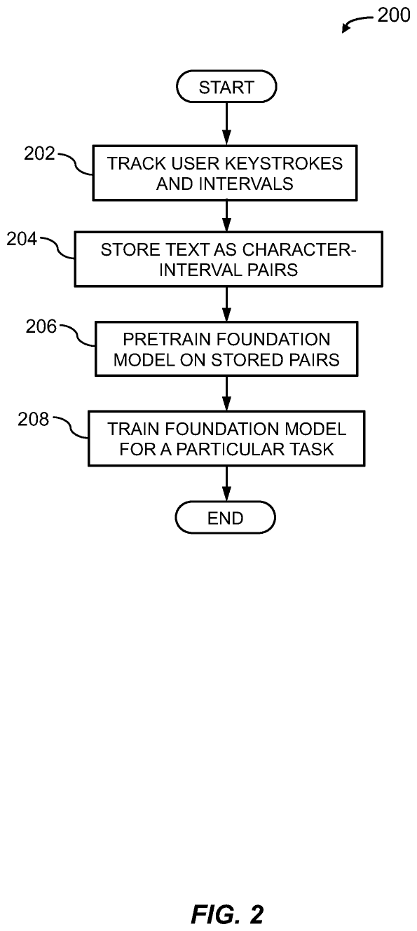

Furthermore, notwithstanding depiction in computer 101, human recognition program 150 may be stored in and/or executed by, individually or in any combination, end user device 103, remote server 104, public cloud 105, and private cloud 106. The event streaming method is explained in more detail below with respect to FIG. 2.

Referring now to FIG. 2, an operational flowchart for a process for human recognition by text analysis 200 is depicted according to at least one embodiment. At 202, the human recognition program 150 tracks user input and intervals between inputs according to opt-in procedures. User input may include keystrokes, touches, characters or inputs in an input stream, clicks, or other forms of input that may be encoded as text. An interval may be a time between inputs, or between like inputs. Tracking input may include tracking additional information, such as the time for which a key is held, the pressure used for a touch input, or a user's mouse settings. Tracking input may further include associating input with other information, including information known about the user, time information, location information, device information, and information about the document or service into which the text is being entered. A user may be a real human, an impostor user purporting to be human such as an algorithm or artificial intelligence, or a test user used to test a part of the process for human recognition by text analysis 200.

In various embodiments, user input may include keystrokes, touches, characters or inputs in an input stream, clicks, or other forms of input that may be encoded as text. For example, user input for users using a physical keyboard may be keystrokes while user input for users using a touch-based keyboard includes a series of touch locations and the associated character input for each touch location. Characters in an input stream may be determined based on keystrokes or touch locations, or taken from an input stream from an operating system's application programming interface (“API”) or a similar service for encoding an input stream. A touch location may be a single touch location, an encoding of multiple touch locations corresponding to multiple touches, or an average of the multiple touch locations.

User input may be collected according to opt-in procedures, and may be collected through any document, text processing application or service, across an operating system, or across all of a user's devices. For example, if a user on a social media network opts into the process for human recognition by text analysis 200, user input may be collected based on all text the user enters into the social media network, or may further include any additional input, consistent with the user's options, that may be useful for the process for human recognition by text analysis 200.

An interval may be a time interval between inputs, or between like inputs. For example, if input includes keyboard input and mouse input, intervals may be the time between one keystroke and another keystroke or one click and another click, or may be the time between one input and the next input, regardless of the nature of the input. An interval between two character inputs may also be referred to as an inter-character input interval (“ICEI”). An interval may be measured in any unit of time, including milliseconds.

In at least one embodiment, tracking input may include tracking additional information, such as the time for which a key is held, the pressure used for a touch input, or a user's mouse settings. For example, tracking may include noting that the shift key is pressed first and held for 1134 milliseconds, that the “T” key is pressed 422 milliseconds after the shift key is initially pressed and held for 9 milliseconds, and the “R” key is pressed 891 milliseconds after the “T” key is initially pressed and held for 17 milliseconds. Alternatively, tracking may include making note that a user has inverted scrolling settings for a trackpad mouse.

In a further embodiment, the human recognition program 150 may identify statistics for a given user, such as a range, mean, median, mode, or standard deviation of intervals for the user, or track statistics about the user typing a particular phrase, such as the three-standard-deviation range of ICEIs measured while the user entered the particular phrase. Tracking standard deviation may be useful, for example, in discerning human users from non-human users, as human users may have a higher variance in ICEIs than many non-human users. As another example, the human recognition program 150 may use range and standard deviation at 208 to train a model to distinguish between two different human users.

In another embodiment, tracking input may further include associating input with other information, including information known about the user, time information, location information, device information, and information about the document or service into which the text is being entered. For example, the human recognition program 150 may identify and track users who are known to have anger issues and identify the times of day during which their anger issues appear to be exacerbated, or track their anger level on a per-application basis, noting that the users are angrier while using or entering text into certain applications than they are using others. Alternatively, the human recognition program 150 may identify and track several test users who are known to have Parkinson's disease and use that knowledge to pretrain or train a machine learning model at 206 or 208 so that it may better recognize the signs of Parkinson's disease, or other signs of aging.

A user may be a real human, an impostor user purporting to be human such as an algorithm or artificial intelligence, or a test user used to test a part of the process for human recognition by text analysis 200. Alternatively, an impostor user maybe a human user claiming to be a different human user, or any user claiming any false attribute, including any attribute that may be tested for with the trained model at 208. An attribute may be, for example, a user's location or purported location, age or purported age, the status or purported status of a user's credential, or any similar trait of a user that may be reflected in the user's text patterns or profile.

Then, at 204, the human recognition program 150 stores the input and intervals in text formatted as input-interval pairs. Inputs and intervals may be any inputs or intervals collected at 202. Input-interval pairs may further include any other data, or be encoded in any other way, so long as the input and intervals are stored for a sufficient duration for use in the process for human recognition by text analysis 200.

In at least one embodiment, input-interval pairs may include a text encoding of a single character representing an input, and another character representing an interval. For example, the human recognition program 150 may store input and intervals in a plain text file consisting of a character in an input stream followed by a character encoding a time in milliseconds representing the interval between the previous input and the next input. A pair may be the first input and the first interval, the second input and the second interval, the third input and the third interval, etc., until the final input and no interval; or the first input and no interval, the first interval and second input, second interval and third input; the third interval and the fourth input, etc. Each input may alternatively be paired with both the interval before and the interval after the input. As another alternative, inputs and intervals may be encoded by more than one character, or in any other way.

In another embodiment, input-interval pairs may be encoded in any data format that is useful for training at 206 or for the process for human recognition by text analysis 200, such as XML and Javascript Object Notation (JSON); in a database; or as an array, list, or object in a programming language.

An input-interval pair may be a simple pair of only the input and the interval, or, alternatively, a combination of these two with any other piece of data. For example, an input-interval set may include two characters signifying the touch coordinates on a touch screen, two more characters signifying the interval, a fifth character signifying the pressure used for the touch, and six more characters representing the approximate location from where the input was provided. In this case, the input-interval pair is represented in the input-interval set by the input (the first, second, and fifth characters) and the interval (the third and fourth characters), and the location data is additional data in the set.

The human recognition program 150 may further store any other data or association collected or identified at 202. Such data and associations may be stored in a variety of ways, such as within each input-interval pair or input-interval set, at the end of a text file containing the input-interval pairs or sets, or in a separate database that is linked to by an object that also contains a list of the input-interval pairs or sets.

In further embodiments, the human recognition program 150 may recognize, analyze, and store data regarding patterns in the data, including n-grams of inputs, words, or characters, intervals between common n-grams, patterns around special inputs such as a backspace or a click, patterns around multiple inputs of the same letter, patterns found by artificial intelligence techniques such as natural language processing, word embedding, content embedding, Fourier spectrum analysis, or any other technique found below, and any interval relevant to any of the above patterns. As another alternative, the human recognition program 150 may recognize patterns in intervals, such as long pauses after a period is entered, and note intervals between such patterns, such as the interval between two long pauses, finding, for example, that a user engages in a pause longer than four seconds at least once in every two-minute period.

Next, at 206, the human recognition program 150 pretrains a foundation model on the stored pairs, sets, or data set as a whole. Pretraining may include training the foundation model using a variety of artificial intelligence and machine learning techniques including natural language processing techniques such as word embedding, content embedding, artificial neural networks, latent space modeling, generative adverse networking, and any other relevant techniques.

Pretraining a foundation model may include training the foundation model to find basic patterns in stored data. For example, pretraining may include masking certain data points in a stored data set, attempting to “predict” or guess the masked data, and training the model based on whether or not each prediction is correct. Such training may use a variety of artificial intelligence and machine learning techniques, including, for example, natural language processing, artificial neural networks, or latent space modeling. Latent space modeling may be particularly useful in modeling or simulating a nervous system of a user.

In at least one embodiment, pretraining may include training the model to perform the particular task at 208. A combined training task may include, for example, simultaneously pretraining the foundation model to predict and find patterns in the data set and training the same model to recognize types of behavior that are and are not thought to be human.

Then, at 208, the human recognition program 150 trains the foundation model to perform a particular task. Training may include use of any of the methods described above at 206 or any other method for training a machine learning model. A particular task may include discerning between a human and non-human user, such as an impostor user; discerning or verifying the identity of a user, or discerning between two human users; emotion analysis or sentiment analysis; detection of motor disorders, injuries, and diseases, or predicting a disease or disorder early; age verification; or any other task for which the data and model may be of assistance. The human recognition program 150 may further use the trained model to perform the task.

A particular task may include discerning between a human and non-human user, such as an impostor user; verifying that a user is a human; or discerning or verifying the identity of a user. Particular tasks may further include age verification or verification or identification of any common trait in a user that could be reflected in text and interval data, verified with varying degrees of confidence, or within a range or cohort. For example, a particular task may include verifying a user's native language, in order to assist with other identity verification software, and may learn from patterns in input-interval pairs, backspaces, and punctuation usage corresponding to the native languages of users.

In another embodiment, the particular task may be emotion analysis or sentiment analysis. In addition to patterns in input-interval pairs, emotion and sentiment analysis may, for example, involve natural language processing of input and learning from patterns in touch pressure. For example, high-speed high-pressure touches may signify anger, whereas high-speed low pressure touches may signify nervousness in certain types of users. The human recognition program 150 may further conduct semantic analysis of the text or use other natural language processing techniques, and correlate data from these techniques with data from analysis of input-interval pairs to conduct a multi-factor emotion analysis or sentiment analysis.

Particular tasks may also include detection of motor disorders, injuries, and diseases, or predicting a disease or disorder early, such as a stroke or Parkinson's disease. The human recognition program 150 may, for example, learn by tracking user input patterns over time, storing input-character intervals according to opt-in procedures, and identifying patterns, symptoms, and early warning signs before the actual onset of certain disorders. Warning signs may be noticed in various time frames depending on the particular disease or disorder. Symptoms and warning signs may include, for example, a slowing from a base typing speed, trouble reaching certain keys in a timely manner, a slowing down of one hand relative to the other hand, an increased prevalence of errors that are then deleted using the backspace key, increased or decreased touch pressure, or holding character keys for a slightly longer or shorter period of time while typing.

The human recognition program 150 may further train a model for any other task for which the data and model may be of assistance.

The human recognition program 150 may train any number of models to perform any number of tasks. For example, the human recognition program 150 may train one copy of the foundation model to discern between different human users and also discern whether or not the users are human, and may train an additional model to recognize each of several disorders or diseases as early as possible, and may further train one model to recognize any of the several disorders or diseases as early as possible.

In further embodiments, the human recognition program 150 may further use the trained model to perform the task. The human recognition program 150 may collect feedback based on the performance of the task, add such feedback to data collected at 202 or stored at 204, and use the feedback to further pretrain or train models.

It may be appreciated that FIG. 2 provides only an illustration of one implementation and does not imply any limitations with regard to how different embodiments may be implemented. Many modifications to the depicted environments may be made based on design and implementation requirements.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method, the method comprising:
tracking user inputs;
tracking time intervals between user inputs;
storing input-interval sets, each input-interval set containing at least one input from the user inputs and at least one time interval from the time intervals between user inputs, wherein the at least one input corresponds to the at least one time interval;
pretraining a foundation model based on the stored input-interval sets;
training the foundation model for a particular task.

2. The method of claim 1, wherein the user inputs are characters in an input stream.

3. The method of claim 1, wherein the time intervals are tracked on a scale of milliseconds.

4. The method of claim 1, wherein the particular task is discerning whether the user is a human.

5. The method of claim 1, wherein tracking user inputs includes tracking user inputs from two or more devices.

6. The method of claim 1, wherein the particular task is identifying or predicting a disease, disorder, or injury.

7. The method of claim 6, further comprising:
performing the particular task; and
using feedback collected about the performance of the particular task in order to further pretrain or further train the foundation model.

8. A computer system, the computer system comprising:
one or more processors, one or more computer-readable memories, one or more computer-readable tangible storage media, and program instructions stored on at least one of the one or more tangible storage media for execution by at least one of the one or more processors via at least one of the one or more memories, wherein the computer system is capable of performing a method comprising:
tracking user inputs;
tracking time intervals between user inputs;
storing input-interval sets, each input-interval set containing at least one input from the user inputs and at least one time interval from the time intervals between user inputs, wherein the at least one input corresponds to the at least one time interval;
pretraining a foundation model based on the stored input-interval sets;
training the foundation model for a particular task.

9. The computer system of claim 8, wherein the user inputs are characters in an input stream.

10. The computer system of claim 8, wherein the time intervals are tracked on a scale of milliseconds.

11. The computer system of claim 8, wherein the particular task is discerning whether or not the user is a human.

12. The computer system of claim 8, wherein tracking user inputs includes tracking user inputs from two or more devices.

13. The computer system of claim 8, wherein the particular task is identifying or predicting a disease, disorder, or injury.

14. The computer system of claim 13, further comprising:
performing the particular task; and
using feedback collected about the performance of the particular task in order to further pretrain or further train the foundation model.

15. A computer program product, the computer program product comprising:
one or more computer-readable tangible storage media and program instructions stored on at least one of the one or more tangible storage media, the program instructions executable by a processor capable of performing a method, the method comprising:
tracking user inputs;
tracking time intervals between user inputs;
storing input-interval sets, each input-interval set containing at least one input from the user inputs and at least one time interval from the time intervals between user inputs, wherein the at least one input corresponds to the at least one time interval;
pretraining a foundation model based on the stored input-interval sets;
training the foundation model for a particular task.

16. The computer program product of claim 15, wherein the user inputs are characters in an input stream.

17. The computer program product of claim 15, wherein the time intervals are tracked on a scale of milliseconds.

18. The computer program product of claim 15, wherein the particular task is discerning whether or not the user is a human.

19. The computer program product of claim 15, wherein tracking user inputs includes tracking user inputs from two or more devices.

20. The computer program product of claim 15, wherein the particular task is identifying or predicting a disease, disorder, or injury.
